(19) United States
(12) Patent Application Publication
(10) Pub. No.: US 2016/0206811 A1
(43) Pub. Date: Jul. 21, 2016

(54) Implantable Electrolytic Diaphragm Pumps

(71) Applicants: Jason Shih, Yorba Linda, CA (US); Andrew Dunn, Santa Monica, CA (US); Changlin Pang, Temple City, CA (US)

(72) Inventors: Jason Shih, Yorba Linda, CA (US); Andrew Dunn, Santa Monica, CA (US); Changlin Pang, Temple City, CA (US)

(21) Appl. No.: 14/982,794

(22) Filed: Dec. 29, 2015

Related U.S. Application Data
(60) Provisional application No. 62/097,973, filed on Dec. 30, 2014.

(51) Int. Cl. A61M 5/142; A61M 5/172; A61M 5/145

(52) U.S. Cl. CPC A61M 5/14276; A61M 5/14593; A61M 5/172; A61F 9/0017

(57) ABSTRACT

In various embodiments, an implantable drug-delivery device features drug-delivery and electrolysis chambers incorporated as separate structures within the device. For example, one or both of these chambers may be manufactured in the form of a bladder that “floats” within the main device housing.

### RELATED APPLICATION

This application claims the benefit of and priority to U.S. Provisional Patent Application No. 62/097,973, filed Dec. 30, 2014, the entire disclosure of which is hereby incorporated herein by reference.

### TECHNICAL FIELD

In various embodiments, the present invention relates generally to implantable pumps for, e.g., drug administration.

### BACKGROUND

Medical treatment often requires the administration of a therapeutic agent (e.g., medicament, drugs, etc.) to a particular part of a patient's body. As patients live longer and are diagnosed with chronic and/or debilitating ailments, the need to place even more protein therapeutics, small-molecule drugs, and other medications into targeted anatomical areas will only increase. Some maladies, however, are difficult to treat with currently available therapies and/or require administration of drugs to difficult-to-reach anatomical regions. Many of these therapies would benefit from concentrated target-area treatment, which would reduce systemic side effects. Furthermore, certain drugs such as protein therapeutics are expensive, costing thousands of dollar per vial. For these reasons, new and improved approaches to targeted drug delivery are constantly sought.

Implantable drug-delivery devices with refillable drug reservoirs address and overcome many of the problems associated with conventional drug-delivery modalities. They generally facilitate controlled delivery of pharmaceutical solutions to a specified target. As the contents of the drug reservoir deplete, a clinician may refill the reservoir in situ, i.e., while leaving the device implanted within the patient's body.

FIG. 1 illustrates an exemplary drug-delivery pump implanted within a patient's eye. The device 100 includes a cannula 102 and a pair of chambers 104, 106 bounded by a first envelope 108. The top chamber 104 defines a drug reservoir that contains the drug to be administered in liquid form, and the bottom chamber 106 contains a liquid which, when subjected to electrolysis using electrolysis electrodes 110, evolves a gaseous product. The two chambers 104, 106 are separated by an expandable (e.g., corrugated) diaphragm 112. The envelope 108 and diaphragm 112 may be made of a biocompatible polymeric material, e.g., parylene.

The cannula 102 connects the top drug reservoir 104 with a check valve 114 inserted at the site of administration or anywhere along the fluid path between the drug reservoir and site of administration. The envelope 108 may reside within a shaped protective shell 116. Control circuitry 118, a battery 120, and an induction coil 122 for power and data transmission are embedded between the bottom wall of the electrolyte chamber 106 and the floor of the shell 116. The device 100 includes one or more ports 124 in fluid communication at least with the drug reservoir 104, which permit a refill needle (not shown) to be inserted therethrough.

Although this design is suited to many applications, alternatives to a configuration in which the electrolysis and drug chambers are different compartments of the same structure and separated by an expandable membrane may be desired. For example, manufacturing considerations or issues of device profile and space utilization may favor separate chambers not part of a single structure (and which may, for example, be separately manufactured).

### SUMMARY

In various embodiments, the present invention relates to electrolytically actuable drug-delivery pumps that incorporate drug-delivery and electrolysis chambers as separate structures within the device. For example, one or both of these chambers may be manufactured in the form of a bladder that “floats” within the main device housing, facilitating use of different materials and separate manufacturing procedures followed by assembly. The various embodiments incorporating separate drug and actuation chambers are applicable not only to electrolysis-actuatable drug-delivery pumps, but also to other actuation mechanisms that are driven by differential pressure, e.g., mechanisms utilizing high vapor-pressure propellant, phase-change materials operative at ambient temperature, reactive materials triggerable by catalyst introduction, etc.

In a first aspect, embodiments of the invention feature an electrolytic drug pump comprising a rigid housing having therein first and second adjacent rigid housing chambers separated by a rigid wall permitting fluid communication between the housing chambers. Within the first housing chamber are a drug reservoir in the form of a bladder comprising a flexible membrane and having an interior fluidically isolated from the first and second housing chambers; a cannula fluidically coupled to the drug reservoir and having an exit port outside the housing; and a refill port fluidically coupled to the drug reservoir and having an entry port outside the housing. Within the second housing chamber are an expandable electrolysis chamber comprising therewithin a plurality of electrolysis electrodes and an electrolysis fluid; a pressure-translating fluid in the first and second housing chambers; and circuitry for operating the electrodes to cause evolution of gas from the electrolysis fluid to thereby expand the electrolysis chamber within the second housing chamber and drive pressure-translating fluid therefrom into the first housing chamber. In this way, the drug reservoir is compressed to drive liquid therein out through the cannula.

In some embodiments, the drug reservoir is defined by the flexible membrane and at least a portion of a side of the rigid wall facing the first chamber, where the flexible membrane has a perimeter joined to the rigid wall. In other embodiments, the flexible membrane is a unitary bladder structure floating within the first housing chamber.

In certain embodiments, the electrolysis chamber is defined by a flexible diaphragm and at least a portion of a side of the rigid wall facing the second chamber, where the flexible diaphragm has corrugations. In other embodiments, the electrolysis chamber is defined by a flexible diaphragm having a bellows structure.

In another aspect, embodiments of the invention feature an electrolytic drug pump comprising a rigid housing having an interior, and within the interior of the housing, (i) a drug reservoir in the form of a unitary expandable bladder structure floating within the housing interior, where the drug reservoir has an interior fluidically isolated from the interior of the housing, and (ii) an expandable electrolysis chamber comprising therewithin a plurality of electrolysis electrodes and an electrolysis fluid. The drug pump further comprises a cannula fluidically coupled to the drug reservoir and having an exit port outside the housing; a refill port fluidically coupled to the drug reservoir and having an entry port outside the housing; and circuitry for operating the electrodes to cause evolution of gas from the electrolysis fluid to thereby expand the electrolysis chamber within the housing and thereby drive a liquid from the drug reservoir out through the cannula.

In various embodiments, the electrolysis electrodes are within the rigid housing, the electrolysis chamber comprising a flexible diaphragm over the electrodes. The flexible diaphragm may be corrugated or uncorrugated, in the form of a bellows, or other suitable configuration. The electrolysis chamber may, if desired, float within the housing interior. The drug pump may include a pressure-translating fluid within the interior of the housing but not within the drug reservoir or the electrolysis chamber.

In still another aspect, embodiments of the invention feature a drug pump comprising a rigid housing having therein first and second adjacent rigid housing chambers separated by a rigid wall permitting fluid communication between the housing chambers. Within the first housing chamber are a drug reservoir in the form of a bladder comprising a flexible membrane and having an interior fluidically isolated from the first and second housing chambers, a cannula fluidically coupled to the drug reservoir and having an exit port outside the housing, and a refill port fluidically coupled to the drug reservoir and having an entry port outside the housing. Within the second housing chamber is an expandable actuation chamber comprising therewithin a pressure-altering mechanism; in other words, the invention is not limited to electrolytic forms of actuation. The drug pump also includes a pressure-translating fluid, responsive to the pressure-altering mechanism, in the first and second housing chambers, whereby operation of the pressure-altering mechanism drives pressure-translating fluid therefrom into the first housing chamber to compress the drug reservoir and thereby drive liquid therein out through the cannula.

In various embodiments, the pressure-altering mechanism comprises or consists of, alone or in combination, an ambient temperature-range phase-change material or a reactive material producing a high vapor pressure and triggerable by catalyst introduction. In the latter case, the drug pump may include circuitry for operating a catalyst-introduction mechanism to cause evolution of gas from the reactive material and thereby expand the electrolysis chamber within the second housing chamber. In this way, pressure-translating fluid is driven therefrom into the first housing chamber, whereby the drug reservoir is compressed to drive liquid therein out through the cannula.

These and other objects, along with advantages and features of the present invention herein disclosed, will become more apparent through reference to the following description, the accompanying drawings, and the claims. Furthermore, it is to be understood that the features of the various embodiments described herein are not mutually exclusive and may exist in various combinations and permutations. As used herein, the terms “approximately” and “substantially” mean ±10%, and in some embodiments, ±5%. The term “consists essentially of” means excluding other materials that contribute to function, unless otherwise defined herein. Nonetheless, such other materials may be present, collectively or individually, in trace amounts.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

FIG. 1 is a schematic sectional view of a prior-art drug-delivery pump implanted at a drug administration site (e.g., in a patient's eye).

FIGS. 2-5 are schematic sectional views showing different configurations of drug pumps in accordance herewith.

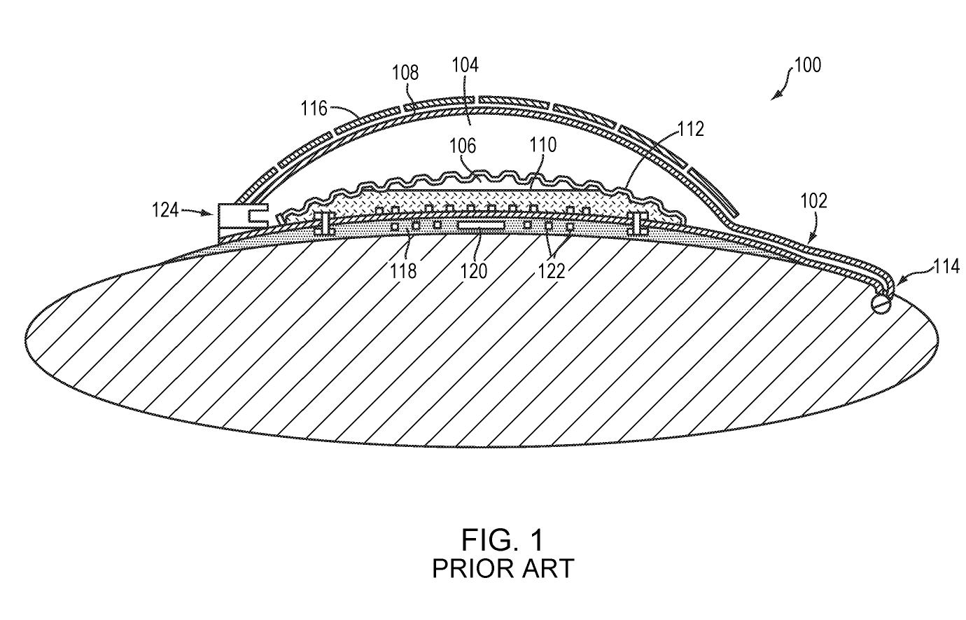

### DETAILED DESCRIPTION

Embodiments of the present invention relate, generally, to implantable drug pump devices that incorporate drug-delivery and electrolysis chambers as separate structures within the device. The devices described herein may be deployed in various internal anatomic regions, including the eye (e.g., between the sclera and conjunctiva), and are suitable as, e.g., implantable insulin pumps, inner ear pumps, and brain pumps.

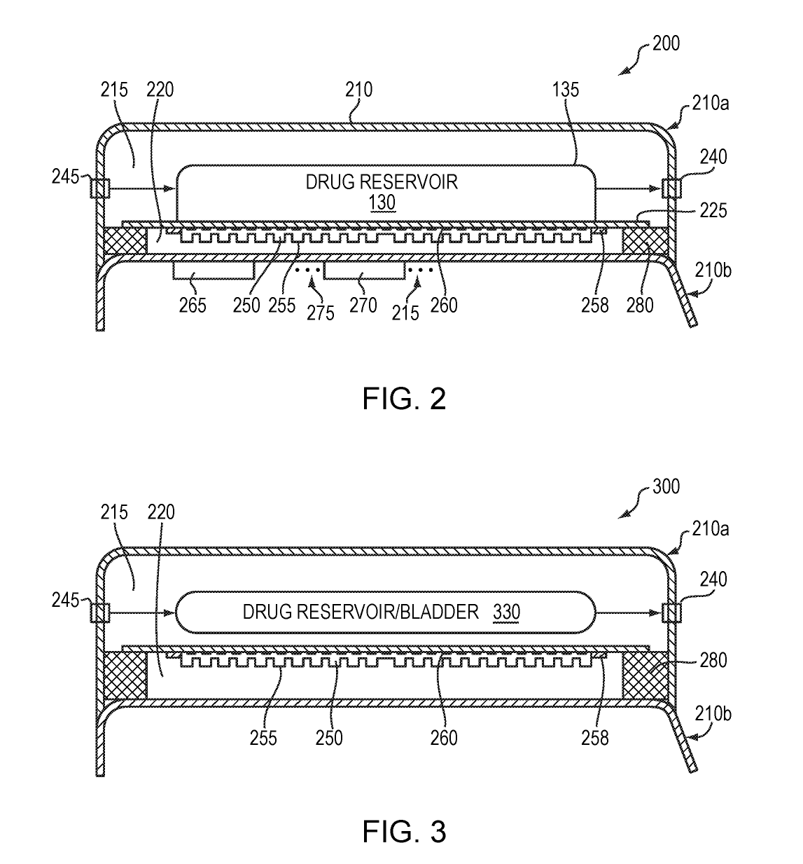

FIG. 2 shows an embodiment 200 in which an outer shell 210 surrounds and defines a pair of internal cavities 215, 220. These cavities are separated by a floor 225, which permits fluid communication between the cavities 215, 220, e.g., by means of one or more perforations. A drug reservoir 230 is disposed within the cavity 215 and is defined by a flexible, elastomeric membrane 235. The drug reservoir 230 is fluidly connected to an exit port 240, to which a cannula can be connected (or, alternatively, a cannula can pass through the shell 210 and join the drug reservoir 230 directly). The shell 210 also desirably includes a refill port 245 that is fluidly connected to the drug reservoir 230 and permits external refill thereof. The membrane 235 is bonded or otherwise affixed around its perimeter to the floor 225, which is non-porous within the region defined by the drug reservoir 230, thereby forming a sealed drug reservoir 230. The refill port 245 may be a polymer tube, a metal via, a pierceable septum, or other fluidic connection that includes a check valve or equivalent functionality. For example, the refill port may have a venting arrangement integrated therewith for, e.g., the venting of excess gas and/or pressure equalization, as described in U.S. patent application Ser. No. 14/317,848, filed Jun. 27, 2014, or U.S. patent application Ser. No. 14/807,940, filed Jul. 24, 2015, the entire disclosure of each of which is hereby incorporated by reference herein.

The cavity 220 contains an electrolysis chamber 250 defined by a diaphragm 255 that is bonded or otherwise affixed around its perimeter to the opposite side of the floor 225. The diaphragm 255 may be elastic and/or may be corrugated, as illustrated, to provide for expansion thereof in response to the phase-change of an electrolysis fluid within the chamber 250 from a liquid to a gaseous state. The diaphragm may alternatively be a bellows structure, i.e., the corrugations or bellows folds form a side wall instead of being distributed across the flat face of the diaphragm. The bellows contains sufficient folds to accommodate the required deflection height. The diaphragm 255 may be manufactured from, for example, one or more parylene films and/or a composite material. Disposed on the surface of the floor 225 that faces the electrolysis chamber 250 are a series of electrodes 260, which, when energized, cause evolution of a gaseous product. These electrodes 260 may be deposited, screen printed, adhered or otherwise applied to the surface of the floor 225, which may, in fact, be an electrolysis chip or other wafer. The electrolyte liquid contained within the electrolysis chamber 250 may include, consist essentially of, or consist of, for example, a saline (i.e., NaCl and H₂O) solution, a solution that contains either magnesium sulfate or sodium sulfate, pure water, or any non-toxic solution. Once again, the floor 225 is non-porous within the region defined by the electrolysis chamber 250, thereby forming a sealed electrolysis chamber. To optimize the mechanics of expansion, the diaphragm 255 may be affixed to a rib 258, which acts as a spacer and prevents contact between the diaphragm 255 and the electrodes 260. In such configurations, the diaphragm 255 is still considered to be joined to the floor whether or not the rib 258 is an integral part of the floor that is, parts herein can be joined directly or indirectly.

Conventional circuitry 265 for operating the electrodes 260, and a battery 270 for powering the circuitry, may be disposed below the bottom of the shell 210. An induction coil 275 for power and data transmission may also be disposed below the bottom of the shell 210. Depending on the complexity of the control functionality it provides, the control circuitry 265 may be implemented, e.g., in the form of analog circuits, digital integrated circuits (such as, e.g., microcontrollers), or programmable logic devices. In some embodiments, the control circuitry 265 controls the pumping action of the pump 100 and includes a microprocessor and associated memory for implementing complex drug-delivery protocols.

The drug pump device 200 may also include various sensors (e.g., pressure and flow sensors) for monitoring the status and operation of the various device components, and such data may be logged in the memory for subsequent retrieval and review. In various embodiments, the induction coil 275 permits wireless (e.g., radio-frequency (RF)) communication with an external controller (e.g., a portable control handset), which may also be used, for example, to charge the battery 270. The coil 275 may be or resemble, for example, a coil described in U.S. patent application Ser. No. 13/491,741, filed on Jun. 8, 2012, the entire disclosure of which is incorporated by reference herein. The external controller may be used to send wireless signals to the control circuitry 265 in order to program, reprogram, operate, calibrate, or otherwise configure the operation of the pump 200. The control circuitry 265 may, for example, communicate electrically with the electrolysis electrodes 260 by means of metal interconnects extending thereto.

Central to the operation of this embodiment is transmission of pressure created in the cavity 220 as the diaphragm 255 expands therewithin to the cavity 215, where it can act on and compress the drug-reservoir membrane 235, thereby driving liquid contained in the drug reservoir out the exit port 240. Accordingly, a pressure-translating fluid fills both cavities 215, 220 and moves freely therebetween through openings in the floor 225. The pressure-translating fluid may be a liquid such as water, or a higher- or lower-viscosity liquid depending on the application. Alternatively, the pressure-translating fluid may be a gas under sufficient pressure to cause force transfer.

The shell 210 may be a single structure, but for ease of manufacture may consist of or comprise two components 210a, 210b. A wafer may be used to form the floor 225 by first depositing or otherwise applying the electrodes 260 thereto (or an already-manufactured electrolysis chip may instead be employed). The spacer rib 258 is either formed with or applied to the surface of the wafer that will receive the diaphragm 255, and both the diaphragm and the membrane 235 are affixed thereto. The floor 225 may be fitted over and secured to a shoulder 280 within the shell component 210a. The shoulder 280 has a thickness corresponding to the desired height of the cavity 220, which is formed when the shell component 210b is affixed to the underside of the shoulder 280. The circuitry, battery and coil may be mounted to the bottom of the shell component 210b before or after it is joined to the component 210a. Both shell components are typically made of a relatively rigid biocompatible material (e.g., medical-grade polypropylene).

After a dose of drug has been dispensed from the drug reservoir 230, the generated electrolysis gas will redissolve in the electrolysis liquid, but the diaphragm 255 may not resume its corrugated, relaxed configuration due to the fixed volume of pressure-translating fluid within the device 200 and the now-smaller volume of the drug reservoir 230. To permit relaxation of the diaphragm 255, the shell 210 may be connected to a separate bladder or made selectively permeable to external bodily fluid via a first check valve to permit the volume of pressure-translating fluid to expand. The check valve ensures that this fluid cannot escape during pump operation and thereby defeat transmission of pressure from the diaphragm 255 to the membrane 235. Such fluid may be expelled back to the separate bladder or an external location via a second check valve with a cracking pressure higher than that of the first check valve; the cracking pressure of the second check valve is achieved during refill of the drug reservoir. This approach may be employed in any of the embodiments described herein. It should be understood that pressure equilibration (and consequent relaxation of the diaphragm) is not essential so long as the additional power needed for actuation can be supplied.

For manufacturing considerations or to permit the use of differently sized drug reservoirs and/or electrolysis chambers, either of these components may “float” within the cavity 215 and/or 220. By “float” is meant that the chamber is a separate unitary structure none of whose surfaces is defined by the shell 200 or the wall 225, and which may in fact be placed rather than adhered therewithin. This permits modular construction of pumps with different configurations that utilize the same validated housing shell 210; for example, a drug reservoir and a corresponding electrolysis chamber may be selected as a matched set from among various paired sizes. Also from the point of view of manufacture, this embodiment minimizes the bonding interface of each reservoir to the interface with the cannula, thereby reducing the number of potential bonding failure points (arising from, e.g., thermal or ultrasonic welding of metal, thermal bonding of parylene, etc.) and in cases of an epoxy bond, minimal exposure of the adhesive to the reservoirs.

FIG. 3 illustrates a configuration 300 in which the drug reservoir 330 takes the form of a bladder whose only mechanical connection to the device 300 may, in some embodiments, be the connections to the ports 240, 245 (or alternatively, in the former case, to a cannula). Each “floating” reservoir can be separately tested and validated prior to assembly of the finished device. (For ease of presentation, the circuitry 265, battery 270 and coil 275 are omitted from FIGS. 2-4.) The drug reservoir 330 may be made of the same material as the membrane 235 illustrated in FIG. 2.

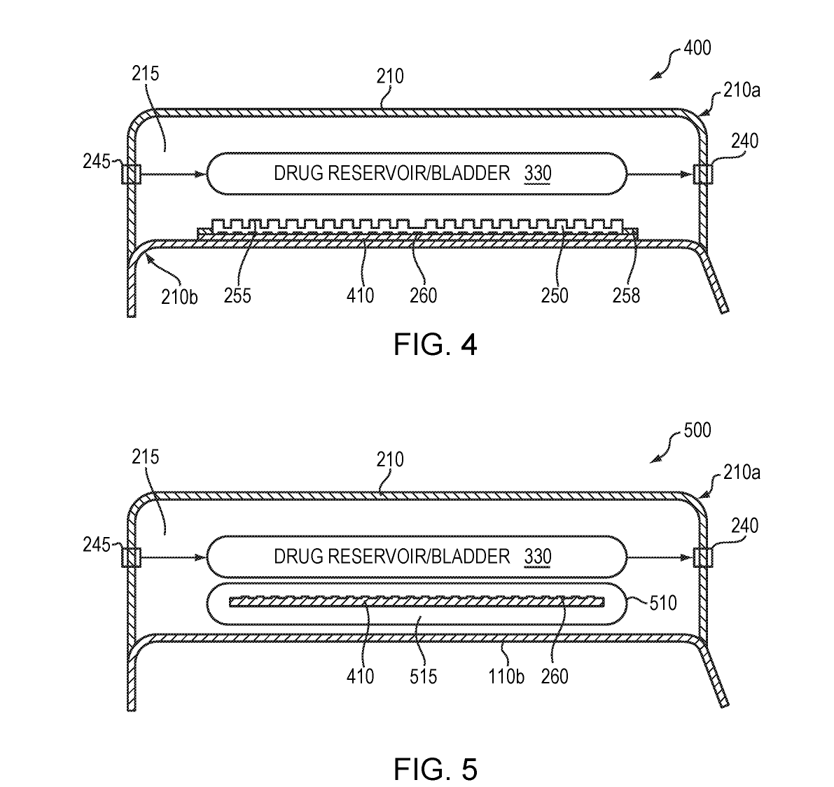

With the drug reservoir in the form of a bladder, it is unnecessary to dispose the electrolysis chamber below the floor 225 in order to avoid a configuration in which the electrolysis chamber lies within the volume of the drug reservoir. Instead, as shown in FIG. 4, the electrolysis chamber 250 may be provided on the surface of the bottom portion 210b of the shell 210. Typically, the floor 410 of the electrolysis chamber 250 will be a wafer with electrodes 260 printed thereon, i.e., an electrolysis chip. The circuitry, battery and coils may be provided on the underside of the shell portion 210b with electrical connections therethrough. Although it is possible to have force transmitted by the inflating electrolysis chamber 250 to the drug reservoir 330 by intimate contact therebetween (and with the top of the drug reservoir 330 pressed against the underside of shell portion 210a), it can be simpler to fill the cavity 115 with a liquid (or pressurized gas) to serve as a pressure-translating medium, thereby ensuring that the displacement of the diaphragm 255 during electrolysis corresponds volumetrically to the amount of drug delivered from the drug reservoir 330.

FIG. 5 illustrates a configuration 500 in which both the drug reservoir 330 and the electrolysis chamber 510 are provided in the form of bladders within the cavity 215. Electrical connection to the electrolysis chip 410 may be provided by wires passing through the envelope of the electrolysis chamber and into the interior 515 thereof. In this configuration, both the drug reservoir 330 and the electrolysis chamber can be made of an elastomeric material such as parylene or silicone. The respective durometers of the materials used for the different chambers may be the same or different, depending on dosing requirements. Again, this permits flexbility in terms of construction with different components to achieve different responses; for example, the same electrolysis chamber 510 may be used with different drug reservoirs 330 having different deformation characteristics and, hence, different dosing responses. Bladder material and reservoir coatings may also be selected to increase shelf life and minimize aggregation of specific drugs (e.g., parylene, titanium internal coatings, etc.)

Separate drug and actuation chambers facilitate the use of other, non-electrolytic actuation mechanisms that are driven by differential pressure, e.g., mechanisms utilizing high vapor-pressure propellant, phase-change materials operative at ambient temperature, reactive materials triggerable by catalyst introduction, etc. Any of these mechanisms can be used to exert force on the pressure-translating medium to thereby compress the drug reservoir and force liquid therein out the exit port 240.

The terms and expressions employed herein are used as terms and expressions of description and not of limitation, and there is no intention, in the use of such terms and expressions, of excluding any equivalents of the features shown and described or portions thereof. In addition, having described certain embodiments of the invention, it will be apparent to those of ordinary skill in the art that other embodiments incorporating the concepts disclosed herein may be used without departing from the spirit and scope of the invention. Accordingly, the described embodiments are to be considered in all respects as only illustrative and not restrictive.

What is claimed is:

1. An electrolytic drug pump comprising:
a rigid housing having therein first and second adjacent rigid housing chambers separated by a rigid wall permitting fluid communication between the housing chambers;
in the first housing chamber, a drug reservoir in the form of a bladder comprising a flexible membrane and having an interior fluidically isolated from the first and second housing chambers;
a cannula fluidically coupled to the drug reservoir and having an exit port outside the housing;
a refill port fluidically coupled to the drug reservoir and having an entry port outside the housing;
in the second housing chamber, an expandable electrolysis chamber comprising therewithin a plurality of electrolysis electrodes and an electrolysis fluid;
a pressure-translating fluid in the first and second housing chambers; and
circuitry for operating the electrodes to cause evolution of gas from the electrolysis fluid to thereby expand the electrolysis chamber within the second housing chamber and drive pressure-translating fluid therefrom into the first housing chamber, whereby the drug reservoir is compressed to drive liquid therein out through the cannula.

2. The electrolytic drug pump of claim 1, wherein the drug reservoir is defined by the flexible membrane and at least a portion of a side of the rigid wall facing the first chamber, the flexible membrane having a perimeter joined to the rigid wall.

3. The electrolytic drug pump of claim 1, wherein the flexible membrane is a unitary bladder structure floating within the first housing chamber.

4. The electrolytic drug pump of claim 1, wherein the electrolysis chamber is defined by a flexible diaphragm and at least a portion of a side of the rigid wall facing the second chamber, the flexible diaphragm having corrugations.

5. The electrolytic drug pump of claim 1, wherein the electrolysis chamber is defined by a flexible diaphragm having a bellows structure.

6. An electrolytic drug pump comprising:
a rigid housing having an interior;
within the interior of the housing:
a drug reservoir in the form of a unitary expandable bladder structure floating within the housing interior, the drug reservoir having an interior fluidically isolated from the interior of the housing; and
an expandable electrolysis chamber comprising therewithin a plurality of electrolysis electrodes and an electrolysis fluid;
a cannula fluidically coupled to the drug reservoir and having an exit port outside the housing;
a refill port fluidically coupled to the drug reservoir and having an entry port outside the housing; and
circuitry for operating the electrodes to cause evolution of gas from the electrolysis fluid to thereby expand the electrolysis chamber within the housing and thereby drive a liquid from the drug reservoir out through the cannula.

7. The electrolytic drug pump of claim 6, wherein the electrolysis electrodes are within the rigid housing, the electrolysis chamber comprising a flexible diaphragm over the electrodes.

8. The electrolytic drug pump of claim 7, wherein the flexible diaphragm is corrugated.

9. The electrolytic drug pump of claim 6, wherein the electrolysis chamber floats within the housing interior.

10. The electrolytic drug pump of claim 9, wherein the flexible diaphragm is corrugated.

11. The electrolytic drug pump of claim 6, further comprising a pressure-translating fluid within the interior of the housing but not within the drug reservoir or the electrolysis chamber.

12. A drug pump comprising:
a rigid housing having therein first and second adjacent rigid housing chambers separated by a rigid wall permitting fluid communication between the housing chambers;
in the first housing chamber, a drug reservoir in the form of a bladder comprising a flexible membrane and having an interior fluidically isolated from the first and second housing chambers;
a cannula fluidically coupled to the drug reservoir and having an exit port outside the housing;
a refill port fluidically coupled to the drug reservoir and having an entry port outside the housing;
in the second housing chamber, an expandable actuation chamber comprising therewithin a pressure-altering mechanism;
a pressure-translating fluid, responsive to the pressure-altering mechanism, in the first and second housing chambers, whereby operation of the pressure-altering mechanism drives pressure-translating fluid therefrom into the first housing chamber to compress the drug reservoir and thereby drive liquid therein out through the cannula.

13. The drug pump of claim 12, wherein the pressure-altering mechanism comprises an ambient temperature-range phase-change material.

14. The drug pump of claim 12, wherein the pressure altering mechanism comprises a reactive material producing a high vapor pressure and triggerable by catalyst introduction.
